(19) United States
(12) Patent Application Publication
(10) Pub. No.: US 2025/0179405 A1
(43) Pub. Date: Jun. 5, 2025

(54) CELL CULTURE SUBSTRATE, CELL CULTURE CONTAINER AND ADHERENT CELL DETACHMENT METHOD

(71) Applicants: Shikoku Instrumentation CO., LTD., Nakatado-gun, Kagawa (JP); SHINSHU UNIVERSITY, Matsumoto City, Nagano (JP)

(72) Inventors: Takeshi Sogou, Nakatado-gun, Kagawa (JP); Yusuke Hirao, Nakatado-gun, Kagawa (JP); Sho Nakazora, Nakatado-gun, Kagawa (JP); Yasushi Tamada, Ueda City, Nagano (JP)

(73) Assignees: Shikoku Instrumentation CO., LTD., Nakatado-gun, Kagawa (JP); SHINSHU UNIVERSITY, Matsumoto City, Nagano (JP)

(21) Appl. No.: 18/843,504

(22) PCT Filed: Sep. 6, 2022
(86) PCT No.: PCT/JP2022/033459

§ 371 Date: Sep. 3, 2024

(30) Foreign Application Priority Data
Mar. 4, 2022 (JP) 2022-033391

(51) Int. Cl. C12M 1/12; C12M 1/00

(52) U.S. Cl. CPC C12M 25/14; C12M 37/00; C12M 47/02

(57) ABSTRACT

Problem: To provide a cell culture substrate, a cell culture vessel, and a cell detachment method for adherent cells, which enable easier detachment of adherent cells compared to a case where trypsin is used. Solution: A cell culture substrate for coating a cell culture vessel, the substrate containing, as an active ingredient, one or more proteins of a silk protein, a silk protein hydrolysate, fibroin, and a fibroin hydrolysate.

### TECHNICAL FIELD

The present invention relates to a cell culture substrate, a cell culture vessel, and a cell detachment method for adherent cells.

### BACKGROUND ART

A technique to collect cultured adherent cells is known in the related art. In this technique, after culture of adherent cells, the adherent cells adhered to a cell culture vessel are detached using trypsin to collect the cultured adherent cells.

In addition, a technique to increase the adhesion of adherent cells to a cell culture vessel is known. In this technique, the cell culture vessel is surface-treated by plasma treatment, gelatin coating, collagen coating, or the like (e.g., Patent Literature 1).

### CITATION LIST

### Patent Literature

Patent Literature 1: JP 05-260950 A

### SUMMARY OF INVENTION

### Technical Problem

In cell detachment treatment of adherent cells in the related art, treatment with trypsin is performed to detach adherent cells from a cell culture vessel. In the trypsin treatment, to prevent components present in a liquid culture medium from reacting with trypsin, steps of removing the liquid culture medium, washing adherent cells with a buffer solution, and then removing the buffer solution used for washing are required before addition of trypsin. Furthermore, since trypsin also causes damage to the surface of cultured adherent cells, after reacting trypsin for a certain time, a step of inactivating trypsin by adding a liquid culture medium to react trypsin with a component in the liquid culture medium is required. Detachment of adherent cells using trypsin as described above takes time and effort, and to reduce production costs, a simpler method for adherent cell detachment is desirable.

Moreover, to increase the adhesion of adherent cells, cell culture vessels that are surface-treated by plasma treatment, gelatin coating, collagen coating, or the like are provided in the related art. However, these surface-treated cell culture vessels are expensive, which is also a factor of increased production costs.

An object of the present invention is to provide a cell culture substrate, a cell culture vessel, and a cell detachment method for adherent cells, which enable easy adherent cell detachment compared to the case where trypsin is used.

### Solution to Problem

A cell culture substrate according to the present invention is a substrate for coating a cell culture vessel and contains, as an active ingredient, one or more proteins of a silk protein, a silk protein hydrolysate, fibroin, and a fibroin hydrolysate.

In the cell culture substrate, the protein functions as a scaffold material for adherent cells and reacts with an enzyme capable of degrading the protein, thus enabling detachment of the adherent cells cultured in the cell culture vessel.

The cell culture substrate can be configured to be sterilized by autoclaving.

A cell culture vessel according to the present invention is coated with the cell culture substrate.

The cell culture vessel can be a configuration in which after application of the cell culture substrate to a hydrophobic vessel for cell culture, the cell culture substrate is insolubilized is performed by further adding a solvent capable of insolubilizing a silk protein, a silk protein hydrolysate, fibroin, or a fibroin hydrolysate, or by using water vapor.

An adherent cell detachment method according to the present invention is a cell detachment method for adherent cells cultured using the cell culture vessel, in which the cultured adherent cells are detached by degrading the protein by an enzyme capable of degrading the protein.

The adherent cell detachment method can be configured to detach the adherent cells by adding the enzyme without washing the cell culture substrate after removal of a liquid culture medium.

The adherent cell detachment method can be configured to detach the adherent cells by adding the enzyme without removal of a liquid culture medium.

### Advantageous Effects of Invention

According to the present invention, adherent cells can be detached from a culture vessel by degrading any one of proteins of: a silk protein; a silk protein hydrolysate; fibroin; and a fibroin hydrolysate; which function as a scaffold material, using an enzyme capable of degrading the protein, and thus the adherent cell detachment can be simply performed without using trypsin.

### BRIEF DESCRIPTION OF DRAWINGS

FIG. 1 is a flowchart for illustrating the procedure of an adherent cell detachment method using trypsin in the related art.

FIG. 2 is a flowchart for illustrating the procedure of an adherent cell detachment method according to the present embodiment.

FIG. 3 includes diagrams for illustrating detachment of adherent cells in a cell culture vessel in the related art and detachment of adherent cell in a cell culture vessel according to the present embodiment.

FIG. 4 includes captured images of petri dishes showing verification results of cell culture in Example 1, in which (A) is an image after culturing adherent cells for 3 days using a petri dish not coated with a cell culture substrate according to the present embodiment, and (B) is an image after culturing adherent cells for 3 days using a petri dish coated with a cell culture substrate according to the present embodiment.

FIG. 5 includes a graph showing a growth curve when adherent cells were cultured by a method according to the present embodiment and a growth curve when adherent cells were cultured by a method in the related art in Example 2.

FIG. 6 includes views showing captured images of adherent cells passaged after culture by a method according to the present embodiment and captured images of adherent cells passaged after culture by a method in the related art in Example 3.

FIG. 7 includes views showing verification results in Example 4.

FIG. 8 includes views showing verification results in Example 5.

FIG. 9 includes views showing verification results in Example 6, the views showing results when an insolubilization treatment with methanol was performed.

FIG. 10 includes views showing verification results when an insolubilization treatment was performed using methanol in Example 6.

FIG. 11 includes views showing verification results when an insolubilization treatment was performed by allowing the cell culture vessel to stand in a water vapor saturated state in Example 6.

FIG. 12 includes views showing verification results when an insolubilization treatment was performed by allowing the cell culture vessel to stand in a water vapor saturated state in Example 6.

FIG. 13 includes views showing verification results of Example 7.

FIG. 14 includes views showing images captured in observation of adherent cells cultured using a cell culture vessel produced by autoclave sterilization of a cell culture substrate according to the present example in Example 8.

FIG. 15 includes views showing images captured in observation of detached states of adherent cells cultured using a cell culture vessel produced by autoclave sterilization of a cell culture substrate according to the present example in Example 8.

FIG. 16 includes views showing verification results of Example 9.

FIG. 17 includes views showing verification results of Example 10, where a cell culture vessel according to the present example was used.

FIG. 18 includes views showing verification results of Example 10, where a cell culture vessel in the related art was used.

FIG. 19 includes views for illustrating verification results of cell culture using a cell culture vessel produced in Example 11.

FIG. 20 includes views for illustrating verification results of cell detachment using a cell culture vessel produced in Example 11.

FIG. 21 includes views for illustrating verification results of cell culture using a cell culture vessel produced in Example 12.

FIG. 22 includes views for illustrating verification results of cell detachment using a cell culture vessel produced in Example 12.

FIG. 23 includes views for illustrating verification results of cell culture using a cell culture vessel produced in Example 13.

FIG. 24 includes views for illustrating verification results of cell detachment using a cell culture vessel produced in Example 13.

### DESCRIPTION OF EMBODIMENTS

Hereinafter, a cell culture substrate, a cell culture vessel, and a cell detachment method according to embodiments of the present invention will be described based on the drawings.

### Cell Culture Substrate

The cell culture substrate according to the present embodiment is a substrate that functions as a scaffold material for increasing the adhesion of adherent cells to a culture vessel and is a substrate that can be used as a coating agent for coating a cell culture vessel. In the present embodiment, the cell culture substrate is characterized by containing, as an active ingredient, a protein that can be degraded by an enzyme, functions as a scaffold material by being deposited onto the bottom surface of a cell culture vessel, and is not possessed by adherent cells. More specifically, the cell culture substrate is characterized by containing a silk protein, a silk protein hydrolysate, fibroin, or a fibroin hydrolysate as an active ingredient.

A silk protein constitutes a type of protein fiber secreted from insects, such as silkworms; or spiders. The form of the silk protein and the degumming method are not particularly limited; not only cocoon thread, raw silk, or silk thread obtained from silkworms or the like can be used as a raw material, but also, for example, a powdery product (ground type) produced by physically grinding them or a hydrolyzed product (hydrolyzed type) produced by hydrolyzing the fiber with acid or alkali can be used as a raw material. In addition, fibroin is a protein constituting a silk protein together with sericin. Fibroin can be extracted using a known method. For example, sericin is a water-soluble protein, and thus fibroin, which is a fibrous protein, can be extracted through removal of sericin by boiling a silk protein in water or by other means. Furthermore, a commercially available silk powder can also be used as fibroin.

In addition, in the present embodiment, the silk protein includes a mixture of fibroin and sericin. The mixture of fibroin and sericin includes a mixture in a state where sericin is not removed by boiling a silk protein as it is in water or the like, and also includes a mixture obtained by adding sericin extracted and purified from a silk protein to a commercially available fibroin or fibroin extracted from a silk protein and purified. The method for producing fibroin and sericin will be described later.

A method for producing the cell culture substrate according to the present embodiment is not particularly limited. The details will be described in Examples below, but for example, a cell culture substrate containing fibroin as an active ingredient can be produced as follows. That is, dried domesticated silkworm cocoons are cut into about 1-cm squares, which are stirred in a heated sodium carbonate solution for about 30 minutes in a heated state to remove sericin. Sodium carbonate is removed, and then the residue is dried to obtain degummed cocoons (fibroin fibers). Then, the resulting degummed cocoons (fibroin fibers) are dissolved to obtain a fibroin solution. Furthermore, the fibroin solution is placed in a dialysis film and dialyzed against deionized water or pure water to remove a salt and a solvent used for dissolution. Furthermore, in the present embodiment, the concentration of fibroin in the fibroin solution after dialysis is adjusted to 0.1 to 1.0%, and a cell culture substrate containing fibroin as an active ingredient can be produced accordingly. In the present embodiment, the concentration of fibroin or the like is expressed as 1% or the like, all of which means wt. %.

In addition, the cell culture substrate according to the present embodiment is not limited to a substrate containing a silk protein, a silk protein hydrolysate, fibroin, or a fibroin hydrolysate as an active ingredient and can also be configured to contain, for example, an egg protein, a milk protein, or a plant protein, such as a soy protein, as an active ingredient. Furthermore, the cell culture substrate according to the present embodiment can be configured to contain water and/or a buffer solution in addition to the protein as the active ingredient. Moreover, the cell culture substrate according to the present embodiment can be configured to contain a mixture of two or more of a silk protein, a silk protein hydrolysate, fibroin, or a fibroin hydrolysate. Still more, the cell culture substrate according to the present embodiment may be configured to further contain a cell adhesion factor, such as fibronectin, collagen, laminin, vitronectin, and pronectin.

Furthermore, the cell culture substrate according to the present embodiment is preferably subjected to a sterilization treatment. Examples of the sterilization treatment include high-pressure steam sterilization by autoclaving, and a sterilization treatment with a γ-ray, an electron beam, or a filter, which has been used for a cell culture substrate in the related art. In addition, the sterilization treatment can also be omitted by using a silk protein, a silk protein hydrolysate, fibroin, a fibroin hydrolysate, or the like sterilized by autoclaving together with a sterilized existing cell culture substrate. It has been confirmed that adherent cells can be suitably grown and detached also in a cell culture substrate produced using a silk protein, a silk protein hydrolysate, fibroin, a fibroin hydrolysate, or the like sterilized by autoclaving.

### Cell Culture Vessel

The cell culture vessel according to the present embodiment is a vessel produced by applying the cell culture substrate according to the present embodiment described above to the bottom surface of an untreated cell culture vessel and coating the bottom with the cell culture substrate. Specifically, a cell culture substrate in a liquid state containing a silk protein, a silk protein hydrolysate, fibroin, or a fibroin hydrolysate as an active ingredient is dropped or sprayed onto the bottom surface of a cell culture vessel with an untreated surface to apply the substrate to all over the bottom surface, and then the bottom surface is dried by placing the vessel in a dryer or by other means. Alternatively, the active ingredient, such as a silk protein, a silk protein hydrolysate, fibroin, or a fibroin hydrolysate, is prevented from dissolving into a liquid culture medium by insolubilization. The insolubilization is performed by adding ethanol or methanol to the bottom surface of the dried cell culture vessel to bring ethanol or methanol into contact with the bottom surface, or by allowing the cell culture vessel to stand for about one night in a water vapor saturated state. After these treatments, the cell culture vessel of the present embodiment may be dried.

In the present embodiment, the type of cell culture vessel is not particularly limited; the cell culture vessel may be provided in the form of, for example, a petri dish, a plate, a flask, or a multilayer flask. In addition, the material of the cell culture vessel according to the present embodiment is not particularly limited, and for example, a vessel made of plastic, glass, or the like can be used. Furthermore, the vessel before being coated with the cell culture substrate according to the present embodiment does not need a special surface treatment, and for example, an inexpensive vessel with an untreated surface (hydrophobic), such as a polystyrene petri dish, can be used. Moreover, in the cell culture vessel according to the present embodiment, not only the bottom surface of the vessel but also the side surface of the vessel may be coated with the cell culture substrate according to the present embodiment. Still more, the cell culture vessel according to the present embodiment is produced using a cell culture substrate in a liquid state, and thus the cell culture vessel can be configured to have a bottom surface or a side surface with a complicated shape.

### Cell Detachment Method

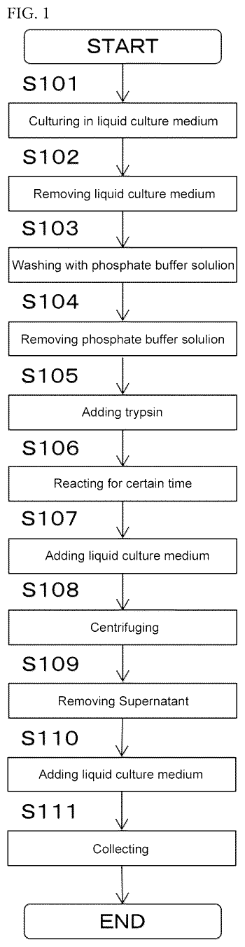

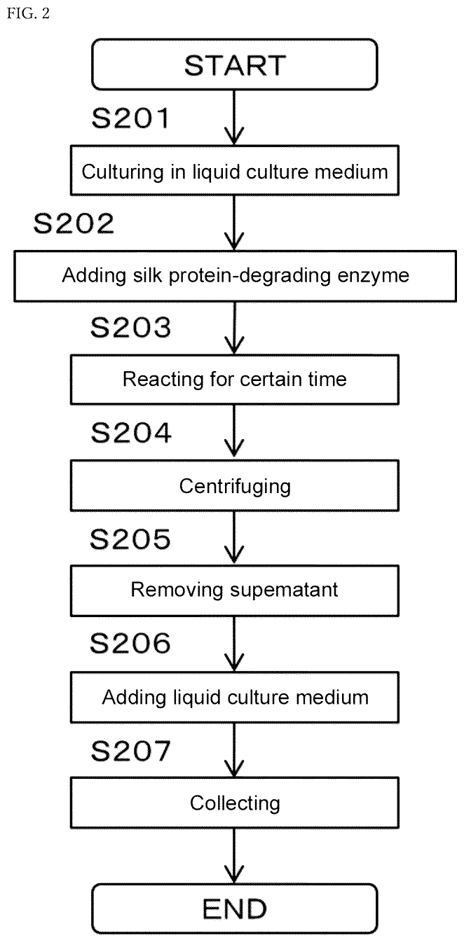

Next, the method for detaching adherent cells cultured using the cell culture vessel according to the present embodiment will be described. First, based on FIGS. 1 and 2, the procedure of a cell detachment method using trypsin in the related art and the procedure of a cell detachment method according to the present embodiment will be described. FIG. 1 is a flowchart for illustrating the procedure of an adherent cell detachment method using trypsin in the related art, and FIG. 2 is a flowchart for illustrating the procedure of an adherent cell detachment method according to the present embodiment.

In the adherent cell detachment method using trypsin in the related art, if trypsin is added without a liquid culture medium being removed, trypsin reacts with a component contained in the liquid culture medium, and adherent cells cannot be sufficiently detached. Thus, after culture of the adherent cells in a cell culture vessel (step S101), a treatment of removing a liquid culture medium is performed (step S102) before addition of trypsin. Furthermore, steps of washing a petri dish with a phosphate buffer solution (step S103) and removing the phosphate buffer solution used for washing (step S104) are also performed. Then, treatments of adding trypsin (step S105) and reacting trypsin at a constant temperature for a certain time (step S106) are performed. In addition, after the trypsin treatment, to suppress damage to the adherent cells caused by trypsin, a treatment of adding a liquid culture medium again (step S107) to inactivate trypsin is performed. Then, the adherent cells are sedimented by centrifugation (step S108), the liquid culture medium as the supernatant is removed (step S109), then a liquid culture medium is added again for storage (step S110), and the sedimented adherent cells can be collected (step S111).

In contrast, the cell culture vessel according to the present embodiment is surface-treated by coating the surface with a silk protein, a silk protein hydrolysate, fibroin, or a fibroin hydrolysate, and the adherent cells are adhered to the cell culture vessel using a silk protein, a silk protein hydrolysate, fibroin, or a fibroin hydrolysate as a scaffold material. Since a silk protein, a silk protein hydrolysate, fibroin, or a fibroin hydrolysate is degraded by reaction with a silk protein-degrading enzyme, the adherent cells can be detached by adding a silk protein-degrading enzyme to a liquid culture medium. In addition, the liquid culture medium does not need to be removed when a silk protein-degrading enzyme that degrades a silk protein, a silk protein hydrolysate, fibroin, or a fibroin hydrolysate that hardly reacts with a protein component contained in a liquid culture medium for the adherent cells is used.

Thus, in a step of detaching adherent cells according to the present embodiment, after culturing adherent cells (step S201), the adherent cells can be detached from the cell culture vessel by adding, as it is, an enzyme that degrades a silk protein, a silk protein hydrolysate, fibroin, or a fibroin hydrolysate (step S202) and reacting the enzyme at a constant temperature for a certain time (step S203) without removing the liquid culture medium, washing with a phosphate buffer solution, and removing the phosphate buffer solution used for washing as in the case of using trypsin illustrated in FIG. 1. In addition, the enzyme that degrades a silk protein, a silk protein hydrolysate, fibroin, or a fibroin hydrolysate acts on the adherent cells themselves to a lesser extent than trypsin. Thus, in the present embodiment, a liquid culture medium need not be added to inhibit the action of the enzyme as in step S107 of FIG. 1 after adding and reacting the enzyme, and the mixture solution is centrifuged as it is (step S204), the supernatant of the liquid culture medium is removed (step S205), then a liquid culture medium is added again for storage (step S206), and the sedimented adherent cells can be collected (step S207).

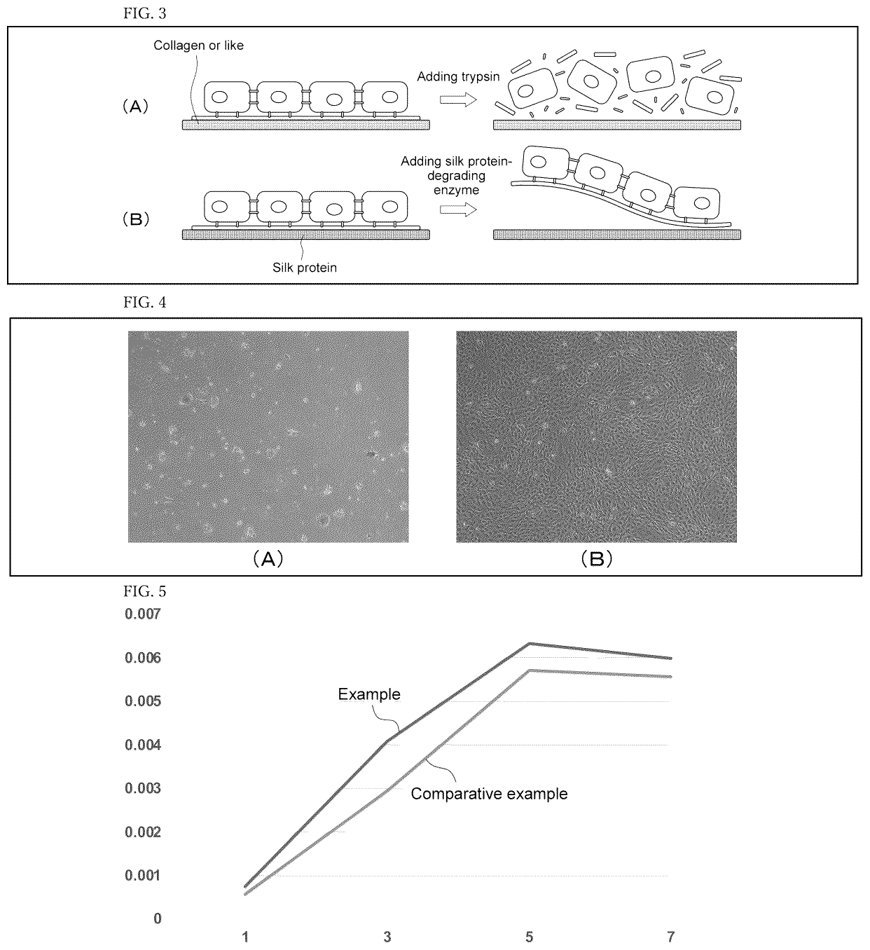

Here, FIG. 3 includes diagrams for illustrating detachment of adherent cells in a cell culture vessel in the related art and detachment of adherent cell in a cell culture vessel according to the present embodiment. FIG. 3(A) illustrates a case where adherent cells cultured in a cell culture vessel in the related art (e.g., a vacuum plasma-treated petri dish) are detached using trypsin, and FIG. 3(B) illustrates a case where adherent cells cultured in the cell culture vessel according to the present embodiment are detached using an enzyme. As illustrated in FIG. 3(A), in the related art, surface treatment by plasma treatment, gelatin coating, collagen coating, or the like is performed on the cell culture vessel to increase the adhesion of adherent cells, and the adherent cells are detached by adding trypsin. However, trypsin degrades not only the bond between the adherent cells but also the bond between the adherent cells and the extracellular matrix as well as the bond between the extracellular matrices, and thus has caused a problem in that the adherent cells come apart as illustrated in FIG. 3(A). In addition, trypsin acts on the adherent cells themselves and has caused a problem of damaging the adherent cells.

In contrast, as illustrated in FIG. 3(B), the cell culture vessel according to the present embodiment is coated with a silk protein, a silk protein hydrolysate, fibroin, or a fibroin hydrolysate, and adherent cells are adhered using the silk protein, the silk protein hydrolysate, fibroin, or the fibroin hydrolysate as a scaffold material. Thus, the adherent cells can be detached from the cell culture vessel by degradation of the silk protein, the silk protein hydrolysate, fibroin, a mixture of fibroin and sericin, or the fibroin hydrolysate using an enzyme that degrades the silk protein, the silk protein hydrolysate, fibroin, the mixture of fibroin and sericin, or the fibroin hydrolysate. In addition, with use of a silk protein-degrading enzyme (e.g., Protease XIV) that specifically acts on a silk protein, a silk protein hydrolysate, fibroin, or a fibroin hydrolysate, the adherent cells do not come apart and can be detached in sheet form as illustrated in FIG. 3(B), and damage to the adherent cells themselves can be suppressed.

### EXAMPLES

Next, examples of cell detachment using the cell culture vessel according to the present embodiment will be described. First, methods for producing a substrate for cell culture and a cell culture vessel used in the present embodiment will be described.

### Production of Substrate for Cell Culture

In Examples 1 to 10 described later, unless otherwise specified, a cell culture substrate containing fibroin as an active ingredient was produced as described below. Specifically, first, dried domesticated silkworm cocoons were cut into about 1-cm squares. Then, 1 L of a 0.02 M (mol/L) sodium carbonate solution was added to 10 g of the dried domesticated silkworm cocoons, the mixture was stirred at 95 to 98° C. for about 30 minutes and sericin was removed from the domesticated silkworm cocoons, and fibroin fibers were obtained. Thereafter, the resulting fibroin fibers were washed with deionized water or ultrapure water to remove sodium carbonate and dried. To 3 g of the dried degummed cocoons (fibroin fibers), 50 mL of a 9 M (mol/L) lithium bromide solution was added to dissolve the degummed cocoons. After adding the lithium bromide solution, the mixture was manually stirred at room temperature of about 25° C. for a certain time. After confirming the dissolution of the degummed cocoons, the mixture was stirred overnight with a stirrer. Then, about 50 mL of the dissolved fibroin solution was placed in a dialysis film (diameter of about 28 mm and a molecular weight cut-off of 12000 to 14000), and a step of dialyzing against 8 L of deionized water or ultrapure water over about half a day was repeated six times. Consequently, lithium bromide in the solution was removed. Furthermore, the concentration of the fibroin aqueous solution after dialysis was measured, and deionized water or ultrapure water was added to adjust the concentration to 0.1 to 1.0%. The concentration of the fibroin aqueous solution after dialysis was measured by a direct gravimetric method (a method of measuring the concentration of the fibroin aqueous solution by determining the weight of the residue (fibroin) obtained by total evaporation of the fibroin aqueous solution after dialysis). Consequently, a cell culture substrate (hereinafter also referred to as the cell culture substrate according to the present example) used in Examples 1 to 10 described below was produced.

In addition, in Examples 11 to 13 described later, a cell culture substrate containing fibroin and sericin as active ingredients or a cell culture substrate containing a fibroin hydrolysate as an active ingredient was produced. Methods for producing cell culture substrates in Examples 11 to 13 will be described in Examples 11 to 13, respectively.

### Production of Cell Culture Vessel

In the present examples, unless otherwise specified, a cell culture vessel coated with a cell culture substrate was produced as follows. Specifically, the cell culture substrate according to the present example was applied onto the inner bottom of a petri dish. The cell culture substrate was applied in an amount such that the entire bottom surface of the vessel was smoothly coated with the substrate. When a large amount of the substrate was applied, an unnecessary portion was removed with a dropper or the like. Then, the petri dish was placed in a dryer and water in the cell culture substrate was evaporated off. Furthermore, after the surface of the petri dish is completely dried, 80% methanol was applied onto the bottom surface of the petri dish to wet the entire bottom surface, and methanol was evaporated at room temperature or in a dryer to perform an insolubilization treatment. Alternatively, instead of insolubilization with 80% methanol, the petri dish was allowed to stand for about one night in a water vapor saturated state to perform insolubilization treatment. The cell culture vessel used in the present examples (hereinafter also referred to as the cell culture vessel according to the present example) was thus produced.

In Examples 1 to 13 below, NIH3T3 was used as the adherent cells. In addition, in Examples 1 to 13 below, unless otherwise specified, the adherent cells were cultured using the cell culture vessel according to the present example produced as described above. Furthermore, in Examples 1 to 13 below, unless otherwise specified, the adherent cells were cultured and detached under the same conditions by the method illustrated in FIG. 2 described above. For the detachment of the adherent cells cultured in the cell culture vessel according to the present example, Protease XIV (available from SIGMA) was used as an enzyme that degrades a silk protein, a silk protein hydrolysate, fibroin, or a fibroin hydrolysate unless otherwise specified.

### Example 1

In Example 1, it was determined whether the adherent cells could be cultured using the cell culture vessel according to the present example. Specifically, as an example, the adherent cells were cultured using the cell culture vessel according to the present example, and as a comparative example, the adherent cells were cultured using a surface-untreated (hydrophobic) polystyrene petri dish not coated with the cell culture substrate according to the present example. FIG. 4 includes views showing verification results of cell culture in Example 1, in which (A) is a captured image of a petri dish after culturing the adherent cells for 3 days using a surface-untreated petri dish not coated with the cell culture substrate according to the present example, and (B) is a captured image of a petri dish after culturing the adherent cells for 3 days using a petri dish coated with the cell culture substrate according to the present example (the cell culture vessel according to the present example). In (B), the cell culture vessel was produced using a cell culture substrate containing 1% of fibroin.

In the example in which the adherent cells were cultured using the cell culture vessel coated with the cell culture substrate according to the present example, growth of the adherent cells was able to be confirmed as shown in FIG. 4(B). In contrast, in the comparative example in which the adherent cells were cultured using a surface-untreated (hydrophobic) polystyrene petri dish not coated with the cell culture substrate according to the present example, growth of the adherent cells could not be confirmed as shown in FIG. 4(A). From this result, it was found that the adherent cells can be grown by using the cell culture vessel according to the present embodiment.

### Example 2

In Example 2, the culture of the adherent cells according to the present example was compared with the culture of adherent cells in the related art to determine whether there would be a difference. Specifically, as an example, after the adherent cells were cultured for different culture times using the cell culture vessel according to the present example, the cultured adherent cells were detached using the enzyme and collected, the activity of lactate dehydrogenase in the collected adherent cell groups were measured, and the number of adherent cells was calculated to create a growth curve in the method according to the present example. In addition, as a comparative example, after the adherent cells were cultured for different culture times using a cell culture vessel in the related art (vacuum plasma-treated cell culture vessel), the cultured adherent cells were detached using trypsin and collected to create a growth curve in the method in the related art. FIG. 5 shows the growth curve in the example in which the adherent cells were cultured by the method according to the present example and the growth curve in the comparative example in which the adherent cells were cultured by the method in the related art. As shown in FIG. 5, growth of the adherent cells to the same extent as that in the related art (rather more than that in the related art) was observed also when the adherent cells were cultured using the cell culture vessel according to the present embodiment, proving that the adherent cells as many as those in the related art (rather more than those in the related art) are obtained.

### Example 3

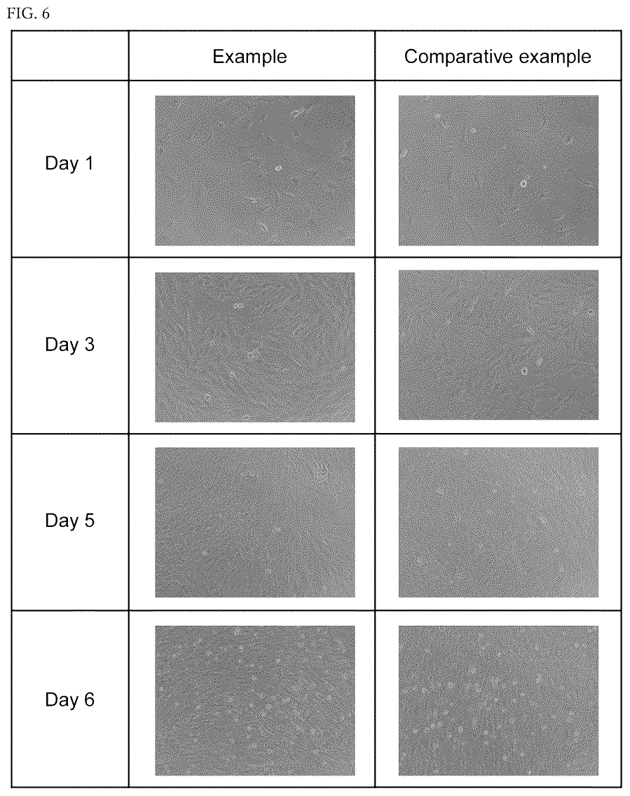

In Example 3, it was determined whether, when the adherent cells cultured using the cell culture vessel according to the present example were passaged, passaged adherent cells could also be cultured in the same manner as before the passage. Specifically, as an example, after the adherent cells were cultured using the cell culture vessel according to the present example, the cultured adherent cells were passaged in a new cell culture vessel according to the present example, and the states of the adherent cells from day 1 to day 6 were observed with a microscope. In addition, as a comparative example, after the adherent cells were cultured using a cell culture vessel in the related art (vacuum plasma-treated cell culture vessel), the cultured adherent cells were newly passaged to the cell culture vessel in the related art, and the states of the adherent cells from day 1 to day 6 were observed with a microscope. FIG. 6 includes views showing the verification results of Example 3 and shows captured images (example) after the passage of the adherent cells cultured by the method according to the present example and captured images (comparative example) after the passage of the adherent cells cultured by the method in the related art. As shown in FIG. 6, even after 6 days from the passage, the growth form after the passage of the adherent cells cultured using the cell culture vessel according to the present example was the same as the growth form after the passage of the adherent cells cultured using the cell culture vessel in the related art, proving that the adherent cells cultured using the cell culture vessel according to the present embodiment can also be appropriately cultured after passage.

### Example 4

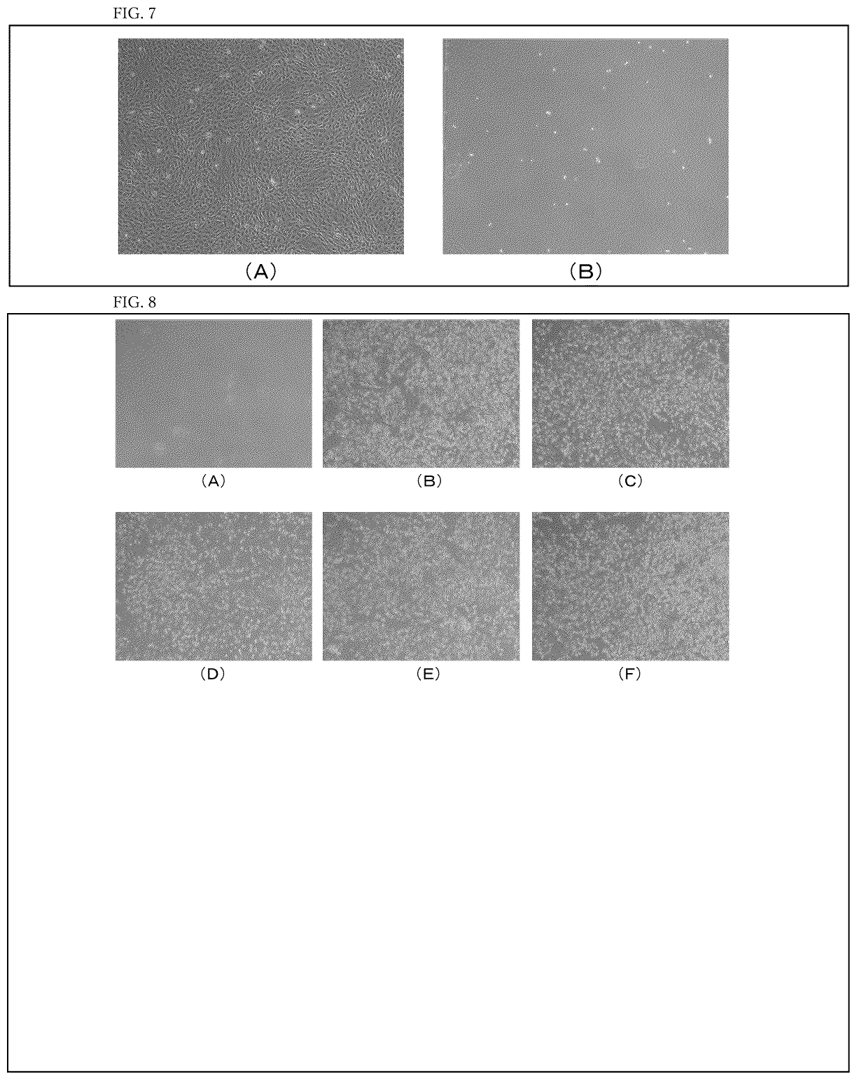

In Example 4, it was determined whether the adherent cells cultured using the cell culture vessel according to the present example could be detached using the enzyme. FIG. 7(A) is a captured image (the same image as that of FIG. 4(B)) of a petri dish after culturing the adherent cells for 3 days using a petri dish coated with the cell culture substrate according to the present example (the cell culture vessel according to the present example) in Example 1, and FIG. 7(B) is a captured image of a petri dish after detachment of the adherent cells of (A) using a 0.1 U/mL Protease XIV. As shown in FIG. 7(B), it was found that the adherent cells cultured using the cell culture vessel according to the present embodiment that was coated with the cell culture substrate according to the present embodiment can be detached by the enzyme (Protease XIV) capable of degrading fibroin.

### Example 5

In Example 5, it was determined whether the adherent cells cultured in a cell culture vessel in the related art (vacuum plasma-treated cell culture vessel) could be detached using the enzyme according to the present example. FIG. 8 includes views showing the verification results in Example 5, in which (A) shows a captured image of a petri dish when the adherent cells cultured in a cell culture vessel in the related art were detached using trypsin, and (B) to (F) are captured images of petri dishes when detachment of the adherent cells cultured in a cell culture vessel in the related art was attempted using the enzyme (Protease XIV). 0.1 U/mL Protease XIV was used in (B), 0.5 U/mL Protease XIV was used in (C), 1.0 U/mL Protease XIV was used in (D), 2.0 U/mL Protease XIV was used in (E), and 5.0 U/mL Protease XIV was used in (F). In addition, in (A) to (F), each enzyme was reacted at 37° C. for 10 minutes. As shown in FIG. 8(A), detachment by trypsin was successful for the adherent cells cultured in the cell culture vessel in the related art, but as shown in FIGS. 8(B) to (F), detachment using Protease XIV was unsuccessful.

### Example 6

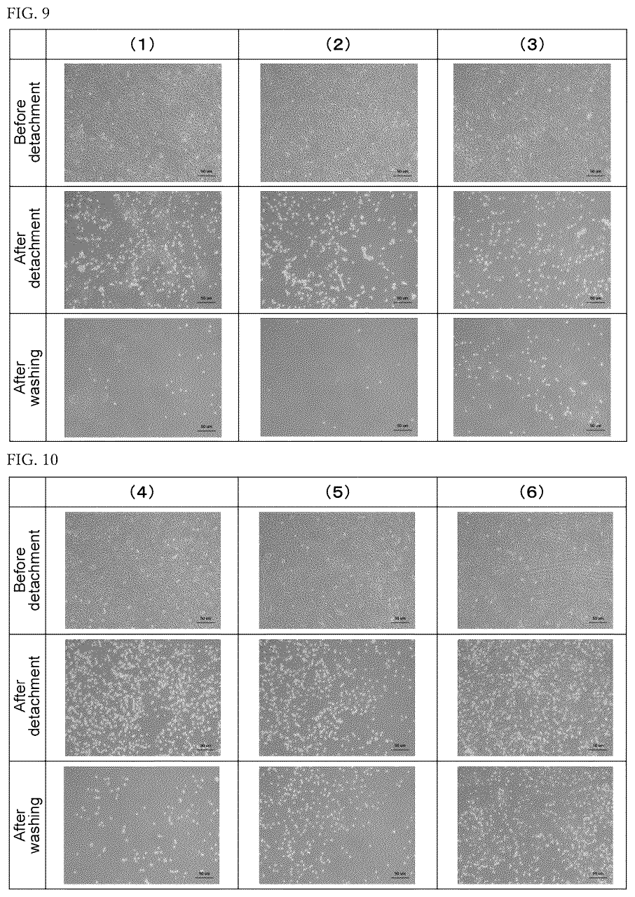

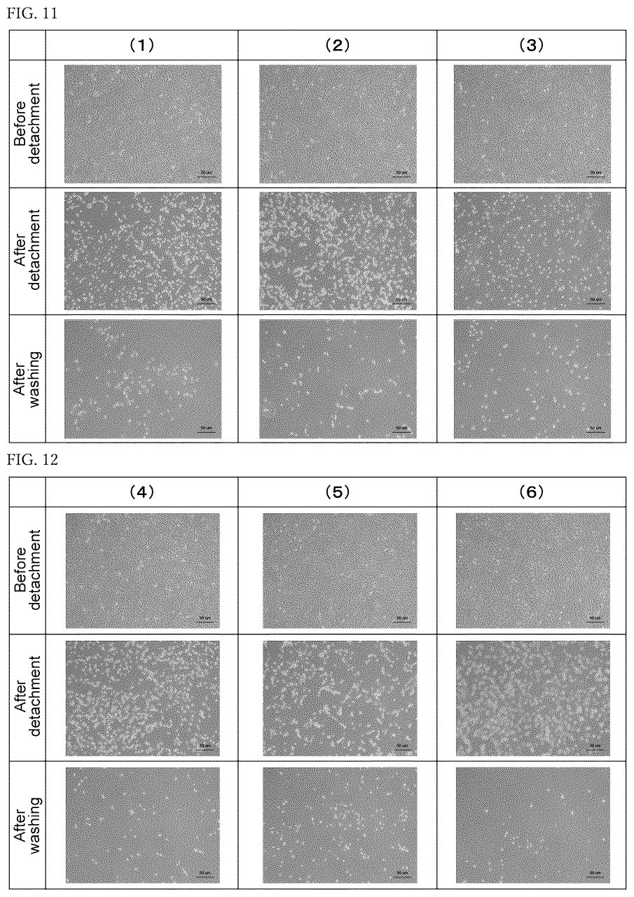

In Example 6, it was determined whether there would be a change in the growth ability and detachability of the adherent cells, depending on the concentration of fibroin in the cell culture substrate according to the present example or depending on the method of the insolubilization treatment in producing the cell culture vessel. Here, FIGS. 9 to 12 are views showing the verification results in Example 6, in which FIGS. 9 and 10 show results when the insolubilization treatment was performed using 80% methanol, and FIGS. 11 and 12 show results when the insolubilization treatment was performed using water vapor. In addition, in Example 6, after culturing the adherent cells for 3 days, the petri dish was washed with a phosphate buffer solution, and then 0.5 mL of 1 U/mL Protease XIV was added to react at 37° C. for 5 minutes, and then the petri dish was washed again with a phosphate buffer solution. In FIGS. 9 to 12, (1) shows results when the concentration of fibroin in the cell culture substrate was 1%, (2) shows results when the concentration of fibroin in the cell culture substrate was 0.5%, (3) shows results when the concentration of fibroin in the cell culture substrate was 0.1%, (4) shows results when the concentration of fibroin in the cell culture substrate was 0.05%, (5) shows results when the concentration of fibroin in the cell culture substrate was 0.01%, and (6) shows results when the concentration of fibroin in the cell culture substrate was 0.005%. Furthermore, in (1) to (6) of FIGS. 9 to 12, the upper images show the state before detachment of the adherent cells by Protease XIV, the middle images show the state immediately after detachment of the adherent cells by Protease XIV, and the lower images show the state after further washing with a phosphate buffer solution after detachment of the adherent cells by Protease XIV.

As shown in FIGS. 9 to 12, it was found that the adherent cells can be appropriately grown and detached when fibroin was present in a concentration of 0.005% or more in the cell culture substrate, and the adherent cells can be more suitably detached when fibroin was present in a concentration of 0.1% or more in the cell culture substrate. In addition, it was also found that the adherent cells can be detached using the enzyme (Protease XIV) regardless of the method of the insolubilization treatment. Here, white spots visible in the captured images after detachment and after washing in FIGS. 9 to 12 are the adherent cells wholly or partially isolated from the cell culture vessel, showing that detachment of the adherent cells has progressed as compared with before detachment.

### Example 7

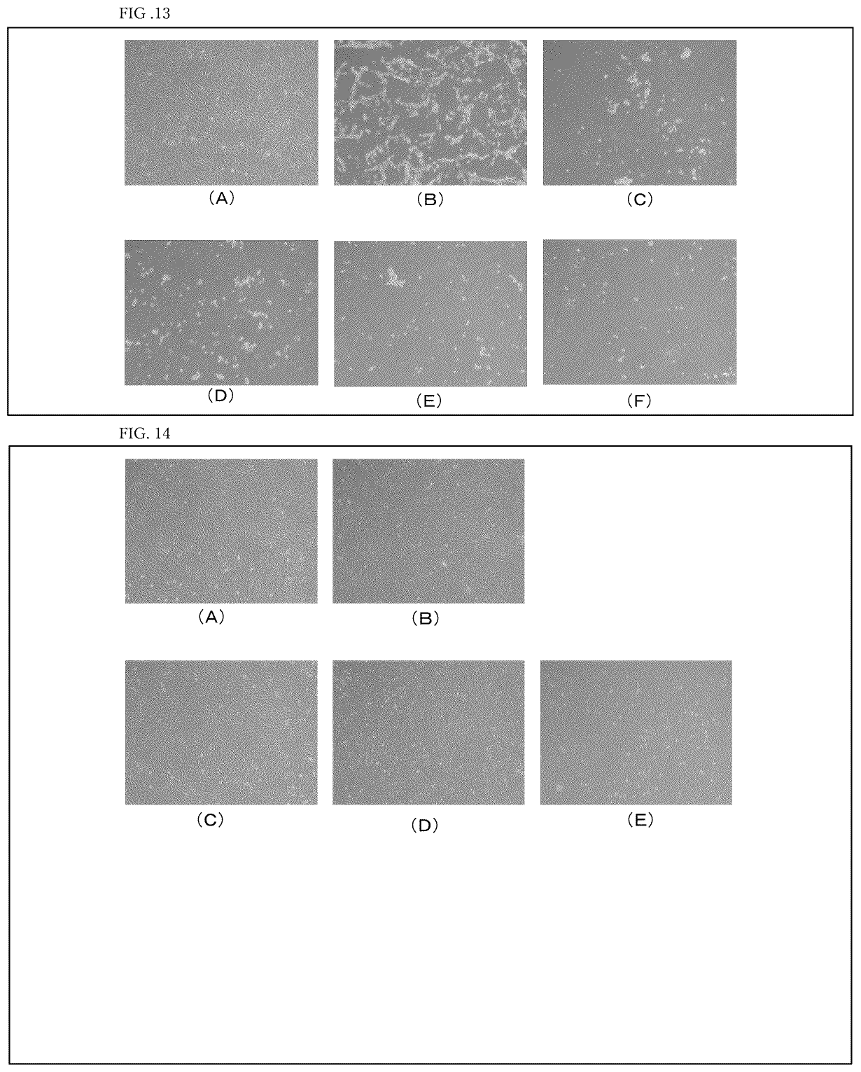

In Example 7, the effect of the concentration of the enzyme on the detachment of the adherent cells was determined. Specifically, the adherent cells were cultured for 3 days using the cell culture vessel in which a cell culture substrate containing 0.1% of fibroin was used and insolubilized with methanol, the liquid culture medium was removed, the vessel was washed with a phosphate buffer solution, and then Protease XIV of each concentration was added and reacted at 37° C. for 5 minutes. FIG. 13 includes views showing the verification results of Example 7. (A) is a view showing the state before detachment by Protease XIV, (B) is a view showing the state after detachment by 0.01 U/mL Protease XIV, (C) is a view showing the state after detachment by 0.05 U/mL Protease XIV, (D) is a view showing the state after detachment by 0.1 U/mL Protease XIV, (E) is a view showing the state after detachment by 0.5 U/mL Protease XIV, and (F) is a view showing the state after detachment by 1.0 U/mL Protease XIV. As shown in FIGS. 13(B) to (F), it was found that the adherent cells can be sufficiently detached by Protease XIV in a concentration of 0.01 U/mL or more. In addition, it was found that the adherent cells can be more suitably detached by Protease XIV of 0.1 U/mL or more as in (D) to (F). Although not shown in the figure, using the cell culture substrate containing 1% fibroin also provided results similar to those produced using the cell culture substrate containing 0.1% fibroin. Furthermore, the insolubilization using water vapor also provided results similar to those obtained by the insolubilization with methanol.

### Example 8

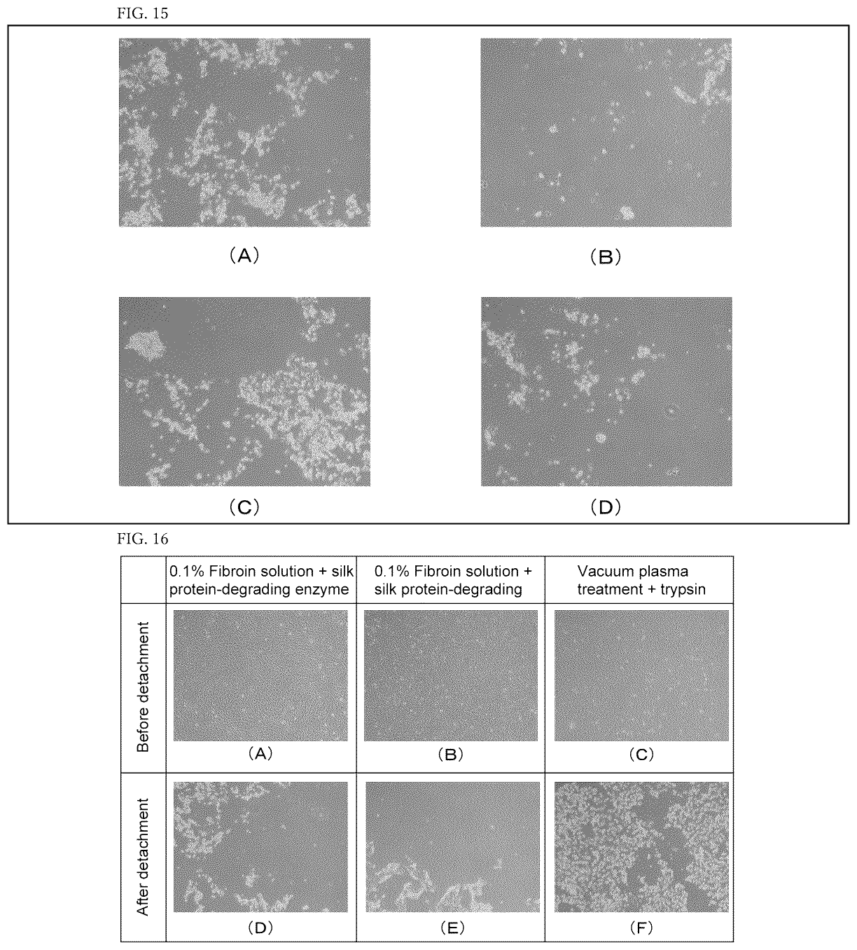

In Example 8, the cell culture substrate according to the present example was sterilized in an autoclave to determine whether the adherent cells could be cultured and detached using a cell culture vessel coated with the cell culture substrate sterilized by autoclaving. Specifically, the cell culture substrate according to the present example was autoclaved at 120° C. for 20 minutes, then the sterilized cell culture substrate was applied onto a sterilized and surface-untreated (hydrophobic) polystyrene plate, dried, and then insolubilized, and a cell culture vessel was produced. Then, the adherent cells were cultured for 4 days using the produced cell culture vessel, and then 0.1 U/mL Protease XIV was reacted for 5 minutes to detach the adherent cells. FIGS. 14 and 15 are views showing the verification results in Example 8, in which FIG. 14 shows images captured in observation of the adherent cells cultured in the cell culture vessel produced using the cell culture substrate according to the present example sterilized in an autoclave, and FIG. 15 shows images captured in observation of detached states of the adherent cells cultured in the cell culture vessel produced using the cell culture substrate according to the present example sterilized in an autoclave.

In FIGS. 14 and 15, (A) shows results when a 0.1% fibroin solution was applied and fibroin was insolubilized with methanol, (B) shows results when a 1% fibroin solution was applied and fibroin was insolubilized with methanol, (C) shows results when a 0.1% fibroin solution was applied and fibroin was insolubilized using water vapor, and (D) shows results when a 1% fibroin solution was applied and fibroin was insolubilized by allowing the cell culture vessel to stand for about one night in a water vapor saturated state. In addition, FIG. 14 (E) shows a result when the adherent cells were cultured on a polystyrene plate for tissue culture in the related art. As shown in FIG. 14, it was found that, similarly to the case on the polystyrene plate for tissue culture in the related art, the adherent cells can be cultured also in the cell culture vessel produced by applying the cell culture substrate (fibroin solution) according to the present embodiment sterilized in an autoclave to a surface-untreated and sterilized vessel as the cell culture vessel. In addition, it was found that using a solution containing 1% or more of fibroin tends to facilitate the detachment as compared with use of a 0.1% fibroin solution. Furthermore, as shown in FIG. 15, it was also found that the cultured adherent cells can be detached by Protease XIV also in the cell culture vessel obtained by applying the cell culture substrate (fibroin solution) according to the present embodiment sterilized in an autoclave to a surface-untreated sterilized vessel as the cell culture vessel. Moreover, although not shown in the figure, also when detachment of the adherent cells was performed by reacting 0.01 U/mL Protease XIV for 5 minutes, the adherent cells were able to be detached. However, it was observed that Protease XIV at 0.1 UmL or more tends to facilitate the detachment of the adherent cells.

### Example 9

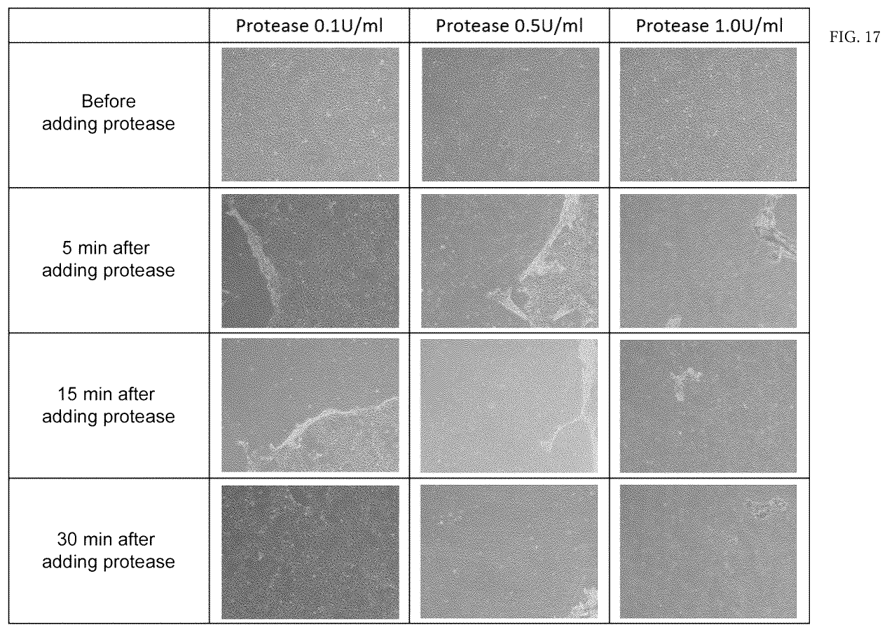

In Example 9, it was determined whether the adherent cells cultured using the cell culture vessel according to the present example could be detached by addition of the enzyme without washing with a phosphate buffer solution after the culture of the adherent cells and removal of the liquid culture medium. Specifically, the cell culture substrate (fibroin solution) according to the present embodiment was sterilized by autoclaving at 120° C. for 20 minutes, and then the fibroin solution was applied onto a surface-untreated (hydrophobic) polystyrene plate. Fibroin was insolubilized, and a cell culture vessel according to the present example was produced. The adherent cells were cultured for 4 days using this cell culture vessel. Thereafter, the liquid culture medium was removed from the cell culture vessel, and 0.1 U/mL Protease XIV was added to the petri dish that remained unwashed with a phosphate buffer solution and reacted for 5 minutes to detach the cultured adherent cells. FIG. 16 shows the verification results of Example 9, in which (A) and (D) show results when a 0.1% fibroin solution was applied and fibroin was insolubilized by allowing the cell culture vessel to stand for about one night in a water vapor saturated state, and (B) and (E) show results when a 1% fibroin solution was applied and fibroin was insolubilized by allowing the cell culture vessel to stand for about one night in a water vapor saturated state. In addition, FIGS. 16(C) and (F) show comparative examples in which the adherent cells cultured on a polystyrene plate for tissue culture in the related art were detached using trypsin without washing the plate with a phosphate buffer solution after removal of the culture solution. As shown in FIGS. 16(C) and (F), the adherent cells cultured on the polystyrene plate for tissue culture were not sufficiently detached even using trypsin when the plate was not washed with a phosphate buffer solution after removal of liquid culture medium, proving that washing with a phosphate buffer solution is necessary. In contrast, as shown in FIGS. 16(A), (B), (D), and (E), it was found that the adherent cells cultured in the cell culture vessel according to the present embodiment can be sufficiently detached by Protease XIV even without washing with a phosphate buffer solution after removal of the liquid culture medium.

### Example 10

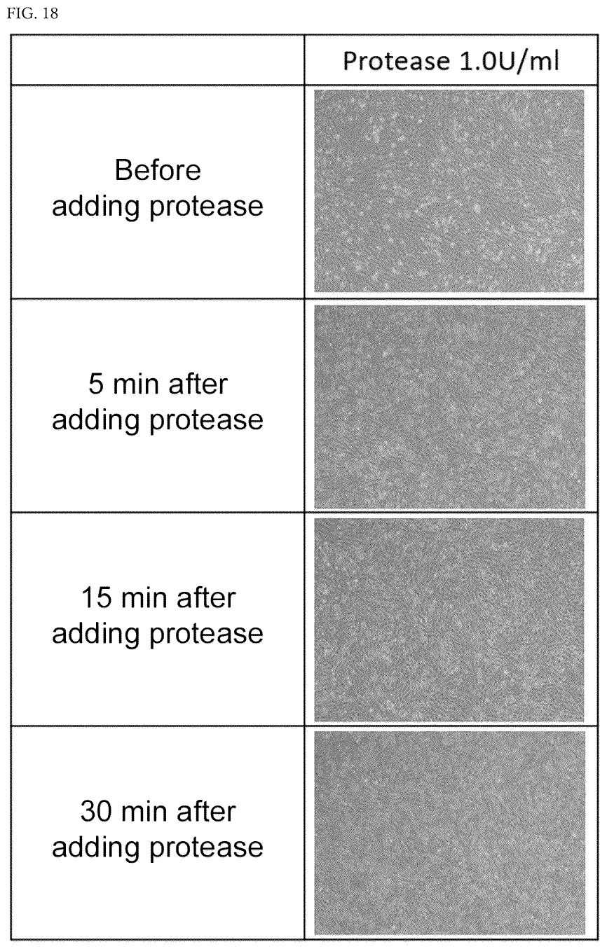

In Example 10, it was determined whether the adherent cells cultured using the cell culture vessel according to the present example could be detached by addition of the enzyme without removal of the liquid culture medium after the culture. Specifically, the cell culture substrate (fibroin solution) according to the present embodiment was sterilized by autoclaving at 120° C. for 20 minutes, and then 1 mL of the fibroin solution was added on a surface-untreated (hydrophobic) polystyrene petri dish (60 mm q). The petri dish was allowed to stand at room temperature for 30 minutes, then the fibroin solution was removed, and the petri dish was allowed to stand overnight in an incubator for insolubilization treatment. Then, the adherent cells were seeded in the produced cell culture vessel (40000 cells/petri dish), and the adherent cells were cultured for 3 days. Thereafter, without removal of the liquid culture medium from the cell culture vessel, Protease XIV was added to the cell culture vessel and reacted for 5 to 30 minutes to detach the cultured adherent cells. FIGS. 17 and 18 are views showing the verification results of Example 10. In the verification shown in FIG. 17, after the adherent cells were cultured using the cell culture vessel according to the present example, Protease XIV was added so that the concentration of Protease XIV in the liquid culture medium was 0.1 U/mL, 0.5 U/mL, or 1.0 U/mL, and the states at 5 minutes, 15 minutes, and 30 minutes after the addition were observed. As shown in FIG. 17, with the addition of Protease XIV in a concentration of 0.1 U/mL or more, some of the adherent cells were detached as if they were turned up at about 5 minutes after the addition. However, at the Protease XIV concentration of 0.1 U/mL, some adherent cells remained adhered without being detached even at 30 minutes after the addition. On the other hand, at the Protease XIV concentration of 0.5 U/mL, many cells were detached at 15 minutes after the addition, and most of the cells were detached at 30 minutes after the addition. At the Protease XIV concentration of 1.0 U/mL, most of the cells were detached at 15 minutes after the addition.

In the verification shown in FIG. 18,after the adherent cells were cultured using a cell culture vessel (vacuum plasma-treated cell culture vessel) in the related art, Protease XIV was added so that the concentration of Protease XIV in the culture medium was 1.0 U/mL without removal of the liquid culture medium, and the states at 5 minutes, 15 minutes, and 30 minutes after the addition were observed. As shown in FIG. 18, when the adherent cells were cultured using the cell culture vessel in the related art and Protease XIV was reacted without removal of the liquid culture medium, the adherent cells could not be detached.

### Example 11

In Example 11, a cell culture vessel was produced using a cell culture substrate with sericin and fibroin mixed. Specifically, fibroin degummed from cocoons was dissolved in a solution obtained by mixing calcium chloride, ethanol, and pure water in a ratio of 8:2:1, and a fibroin solution containing 1 wt. % of fibroin was produced. In addition, about 20 mL of pure water was added to 0.25 g of cocoons, and the mixture was heat-treated at 120° C. for 20 minutes for extraction, and a sericin aqueous solution containing 1 wt. % of sericin was produced. Then, the fibroin solution and the sericin aqueous solution were mixed in weight ratios of 7:3, 5:5, 3:7, and 0:10 to prepare respective mixed solutions. The prepared mixed solutions were sterilized by autoclaving at 120° C. for 20 minutes, and cell culture vessels according to Example 11 were produced by applying each mixed solution to a petri dish and then drying overnight at 50° C. In Example 11, an insolubilization treatment was not performed. Then, the adherent cells (NIH3T3 cells) were cultured for 4 days using the completed cell culture vessel.

FIG. 19 shows captured images of the petri dishes after culturing the adherent cells using the cell culture vessel according to Example 11. As shown in FIG. 19, it was found that the adherent cells can be cultured in all the cell culture vessels produced using the cell culture substrates produced by mixing the fibroin solution and the sericin aqueous solution in weight ratios of 7:3, 5:5, 3:7, and 0:10. Next, after the culture medium was removed from these four cell culture vessels, 1 U/mL of Protease XIV was added, and the state of detachment of the adherent cells was observed after 5 to 7 minutes elapsed. As shown in FIG. 20, it could be confirmed from the results that in the cell culture substrates produced by mixing the fibroin solution and the sericin aqueous solution in 7:3, 5:5, and 3:7, the cells can be detached in sheet form. On the other hand, in the cell culture vessel produced using the cell culture substrate containing no fibroin solution and containing only sericin (the cell culture substrate produced by mixing the fibroin solution and the sericin aqueous solution in 0:10), cell detachment could not be confirmed. It was thus found that cell detachment using protease can also be achieved with the cell culture substrate produced by mixing the sericin aqueous solution and the fibroin solution. FIG. 20 includes views for illustrating the verification results of cell detachment in Example 11 and shows captured images of the petri dishes after detachment of the adherent cells cultured in the cell culture vessels according to Example 11. In addition, in FIG. 20, for the cell culture substrate produced by mixing the fibroin solution and the sericin aqueous solution in 0:10, only a photographic image at a high magnification is shown.

### Example 12

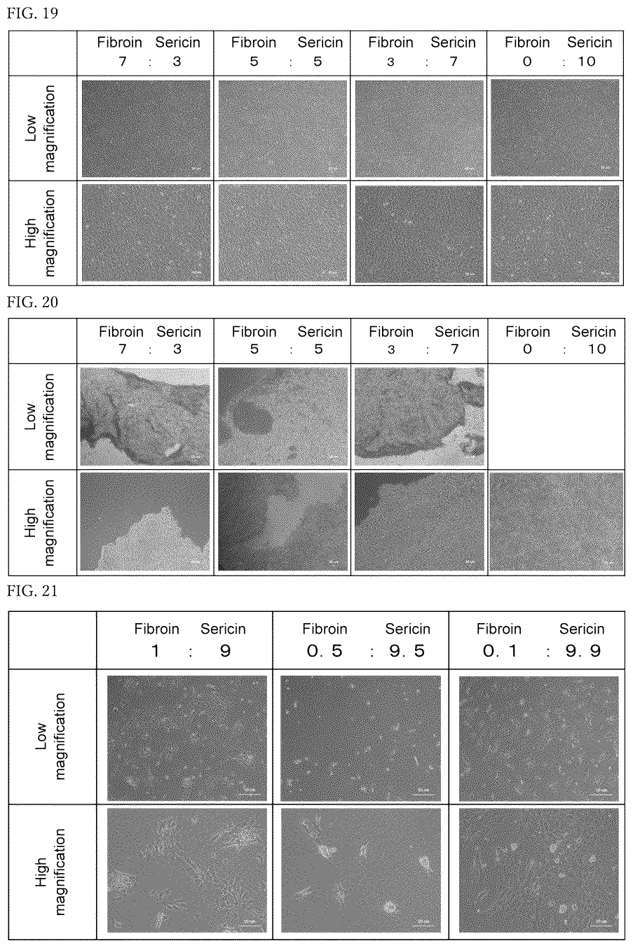

In Example 11 described above, the adherent cells were able to be grown in the cell culture vessel produced using the cell culture substrate containing no fibroin and containing only sericin, but it was found that the adherent cells cannot be detached from the cell culture vessel using protease. Thus, in Example 12, the required amount of fibroin (the proportion of fibroin required) was examined for a cell culture substrate with sericin and fibroin mixed. Specifically, in Example 12, a cell culture substrate was prepared by mixing a fibroin solution and a sericin aqueous solution in weight ratios of 1:9, 0.5:9.5, and 0.1:9.9, respectively. The methods for preparing the fibroin solution and the sericin aqueous solution were the same as in Example 11. Then, the mixed solutions were sterilized by autoclaving at 120° C. for 20 minutes, and cell culture vessels according to Example 12 were produced by applying each mixed solution to a petri dish. After application of the cell culture substrate to the cell culture vessel, the cell culture vessel was dried overnight at 50° C., and an insolubilization treatment was not performed. Then, the adherent cells (NIH3T3 cells) were cultured for 3 days using the produced cell culture vessels. FIG. 21 shows captured images of the petri dishes after culturing the adherent cells using each of the cell culture vessels of Example 12. As shown in FIG. 21, the adherent cells were successfully cultured in all the cell culture vessels produced using the cell culture substrates containing the mixed solution produced by mixing the fibroin solution and the sericin aqueous solution in 1:9, 0.5:9.5, and 0.1:9.9, but the adhesion of the adherent cells to the cell culture substrate tended to be poor as compared with Example 11.

Next, after the culture medium was removed from these cell culture vessels, 1 U/mL of Protease XIV was added, and the state of cell detachment was observed after 5 minutes elapsed. As a result, as shown in FIG. 22, detached adherent cells appeared to be floating and round in the cell culture vessels produced using the cell culture substrates produced by mixing the fibroin solution and the sericin aqueous solution in 1:9 and 0.5:9.5, and it could also be confirmed by observation with the naked eyes that the adherent cells can be detached in sheet form. On the other hand, as shown in FIG. 22, the adherent cells appeared to be spread due to adhesion in the cell culture vessel produced using the cell culture substrate produced by mixing the fibroin solution and the sericin aqueous solution in 0.1:9.9, and detachment of the adherent cells could not be confirmed. FIG. 22 includes views for illustrating the verification results of cell detachment in Example 12 and shows captured images of the petri dishes after detachment of the adherent cells cultured in the cell culture vessels of Example 12.

### Example 13

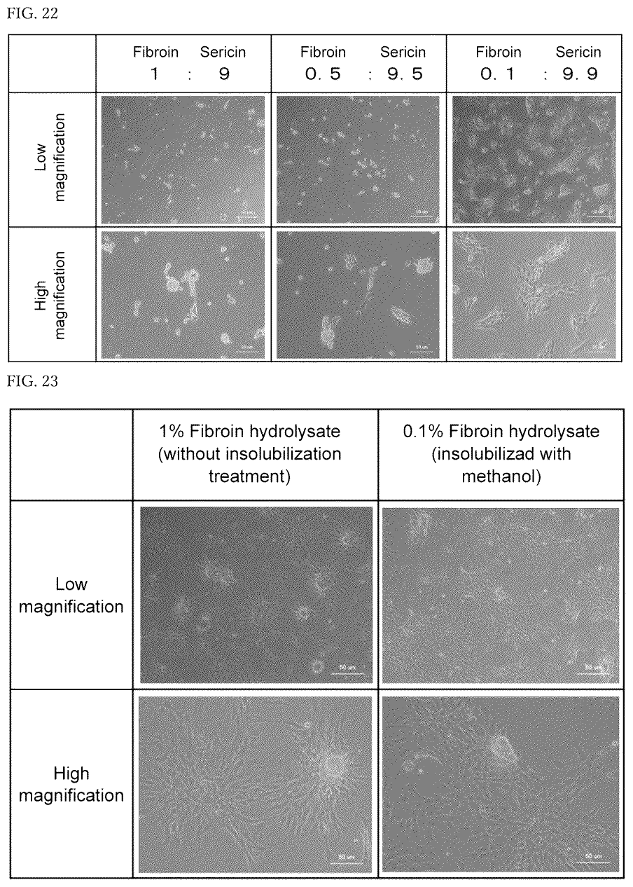

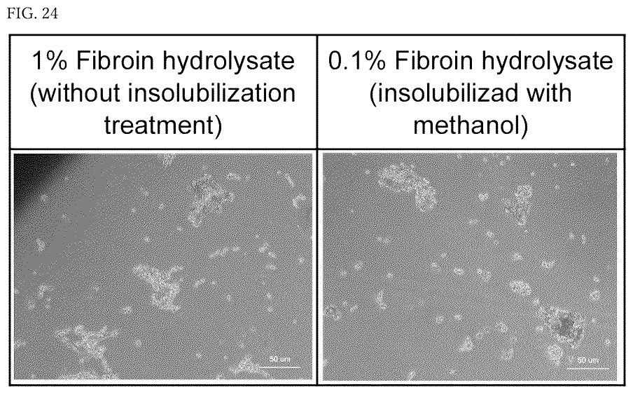

In Example 13, the adherent cells were cultured and detached using cell culture vessels containing a cell culture substrate containing a fibroin hydrolysate produced by hydrolysis of fibroin as an active ingredient. Specifically, sodium hydroxide was added to a fibroin aqueous solution containing 8 wt. % of fibroin so that sodium hydroxide was 2 N, and hydrolysis was performed at 60° C. for 1 hour. Furthermore, the resulting hydrolysate was neutralized with hydrochloric acid and dialyzed to prepare a hydrolyzed fibroin aqueous solution. Next, an aqueous solution of a fibroin hydrolysate was sterilized by autoclaving at 120° C. for 20 minutes, and cell culture substrates each with a concentration in terms of fibroin of 1 wt. % and 0.1 wt. % were produced. Then, the produced cell culture substrates were each applied to a cell culture vessel and then dried overnight at 50° C. For the cell culture vessel produced using the cell culture substrate with a concentration in terms of fibroin of 0.1 wt. %, the hydrolysate of fibroin was insolubilized with 80% methanol, and for the cell culture vessel produced using the cell culture substrate with a concentration in terms of fibroin of 1 wt. %, the hydrolysate of fibroin was not insolubilized. Then, the adherent cells (NIH3T3 cells) were cultured for 3 days using each of the produced cell culture vessels. FIG. 23 shows captured images of the petri dishes after culturing the adherent cells using each of the cell culture vessels produced in Example 13. As shown in FIG. 23, the adherent cells were successfully cultured in all the cell culture vessels produced using the cell culture substrates containing an aqueous solution of a fibroin hydrolysate, but a variation was observed in the growth of the adherent cells as compared with other examples. In addition, after the culture medium was removed, 1 U/mL of Protease XIV was added, and the state of cell detachment was observed after 5 minutes elapsed. As a result, as shown in FIG. 24, detached adherent cells appeared to be floating and round also in the cell culture vessels produced using the cell culture substrate containing the fibroin hydrolysate as an active ingredient, confirming that the adherent cells can be detached. FIG. 24 includes views for illustrating the cell detachment using the cell culture vessels produced in Example 13 and shows captured images of the petri dishes after detachment of the adherent cells cultured in each of the cell culture vessels of Example 13. As shown in FIG. 24, the adherent cells were successfully cultured and detached also with the cell culture substrate containing the fibroin hydrolysate, but for the culture of the adherent cells, other examples achieved preferred results.

As described above, the cell culture substrate according to the present embodiment is a substrate for coating a cell culture vessel and contains, as an active ingredient, one or more proteins of a silk protein, a silk protein hydrolysate, fibroin, and a fibroin hydrolysate. Consequently, one or more proteins of a silk protein, a silk protein hydrolysate, fibroin, and a fibroin hydrolysate function as a scaffold material for adherent cells in a cell culture vessel coated with the cell culture substrate according to the present embodiment, and cultured adherent cells can be detached by degrading one or more proteins of a silk protein, a silk protein hydrolysate, fibroin, and a fibroin hydrolysate by an enzyme capable of degrading one or more proteins of a silk protein, a silk protein hydrolysate, fibroin, and a fibroin hydrolysate. In particular, a silk protein, a silk protein hydrolysate, and fibroin are proteins widely used in the cosmetic field and the medical field and can be produced at low cost, and a cell culture vessel coated with the cell culture substrate according to the present embodiment can be produced at low cost.

In addition, autoclave sterilization does not affect the capability of the cell culture substrate according to the present embodiment to culture and detach adherent cells, and the cell culture substrate can be provided in a sterile state. Furthermore, after application of the cell culture substrate to a vessel, a silk protein, a silk protein hydrolysate, fibroin, or a fibroin hydrolysate contained in the cell culture substrate can be immobilized on the vessel by performing an insolubilization treatment by further adding a solvent capable of insolubilizing a silk protein, a silk protein hydrolysate, fibroin, or a fibroin hydrolysate, or by using water vapor.

Furthermore, a silk protein-degrading enzyme that specifically acts on a silk protein, a silk protein hydrolysate, fibroin, or a fibroin hydrolysate is used. This allows the silk protein-degrading enzyme to react with a silk protein, a silk protein hydrolysate, fibroin, or a fibroin hydrolysate without being deactivated by a protein component in the liquid culture medium and can also suppress the silk protein-degrading enzyme from reacting with the extracellular matrix and/or adherent cells themselves as compared with trypsin. Thus, this eliminates the need for the step of removing the liquid culture medium (step S102 in FIG. 1), the step of washing with a phosphate buffer solution (step S103 in FIG. 1), the step of removing the phosphate buffer solution used for washing (step S104 in FIG. 1), and the step of adding the liquid culture medium again to suppress damage to adherent cells caused by trypsin (step S107 in FIG. 1), which have been required for detachment treatment of adherent cells using trypsin, and allows simplification and speed-up of the process and cost reduction.

Furthermore, for the cell culture substrate according to the present embodiment, an enzyme that is less likely to react with the extracellular matrix and/or adherent cells themselves is used, and thus the adherent cells after detachment do not come apart and can also be obtained in sheet form.

Although preferred embodiment examples of the present invention have been described above, the technical scope of the present invention is not limited to the description of the above embodiments. Various modifications and/or improvements can be made to the above embodiment examples, and an embodiment to which such a modification or an improvement is made is also included in the technical scope of the present invention.

For example, in the embodiments described above, the cell culture substrate according to the present invention has been described by exemplifying a substrate in a liquid state or a semi-liquid state containing a silk protein, a silk protein hydrolysate, fibroin, or a fibroin hydrolysate as an active ingredient, but the cell culture substrate is not limited thereto and can be provided as, for example, a sheet-like or film-like substrate. In this case, the cell culture vessel according to the present invention can be produced by attaching a sheet-like or film-like cell culture substrate to the surface of a petri dish.

In addition, in the embodiments described above, the process of detaching adherent cells has been exemplified by a process without removal of a liquid culture medium, washing with a phosphate buffer solution, and removal of the phosphate buffer solution used for washing. However, the process of detaching adherent cells is not limited to this configuration and may be a process, for example, in which adherent cells cultured using the cell culture vessel according to the present embodiment are detached by removing a liquid culture medium, washing with a phosphate buffer solution, and removing the phosphate buffer solution used for washing.

Furthermore, in Example 11 described above, the mixed solution of fibroin and sericin was prepared by mixing the sericin aqueous solution of sericin extracted from cocoons to the fibroin solution produced using fibroin degummed from cocoons. However, the preparation is not limited to this configuration, and the mixed solution of fibroin and sericin can be configured to be prepared by mixing a sericin aqueous solution extracted from cocoons to a fibroin solution produced using a commercially available fibroin. Alternatively, the mixed solution of fibroin and sericin may be configured to be prepared by boiling a silk protein as it is in water. In this case, fibroin and sericin are contained in a weight ratio of 7:3 to 8:2.

1. A cell culture substrate for coating a cell culture vessel, the substrate comprising, as an active ingredient, one or more proteins of a silk protein, a silk protein hydrolysate, fibroin, and a fibroin hydrolysate.

2. The cell culture substrate according to claim 1, wherein the protein functions as a scaffold material for adherent cells and reacts with an enzyme capable of degrading the protein, thus enabling detachment of the adherent cells cultured in the cell culture vessel.

3. The cell culture substrate according to claim 1, wherein the cell culture substrate is sterilized by autoclaving.

4. A cell culture vessel coated with the cell culture substrate described in claim 1.

5. The cell culture vessel according to claim 4, wherein after application of the cell culture substrate to a hydrophobic vessel for cell culture, the cell culture substrate is insolubilized is performed by further adding a solvent capable of insolubilizing a silk protein, a silk protein hydrolysate, fibroin, or a fibroin hydrolysate, or by using water vapor.

6. A cell detachment method for adherent cells, the method being used for detachment of adherent cells cultured using the cell culture vessel described in claim 4, wherein
the cultured adherent cells are detached by degrading the protein by an enzyme capable of degrading the protein.

7. The cell detachment method for adherent cells according to claim 6, wherein after removal of a liquid culture medium, the adherent cells are detached by adding the enzyme without washing the cell culture substrate.

8. The cell detachment method for adherent cells according to claim 6, wherein the adherent cells are detached by adding the enzyme without removal of a liquid culture medium.
